(19) United States
(12) Patent Application Publication
(10) Pub. No.: US 2013/0160761 A1
(43) Pub. Date: Jun. 27, 2013

(54) Pharmaceutical Aerosol Composition

(71) Applicants: CIPLA LIMITED, Mumbai (IN); Mustafa Khwaja Hamieel, Mumbai (IN)

(72) Inventors: Amar LULLA, Mumbai (IN); Geena MALHOTRA, Mumbai (IN); Rajendra Narayanrao KANKAN, Mumbai (IN); Dharmaraj Ramachandra RAO, Mumbai (IN); Maruti GHAGARE, Thane (W) (IN)

(73) Assignee: CIPLA LIMITED, Mumbai (IN)

(21) Appl. No.: 13/685,949

(22) Filed: Nov. 27, 2012

Related U.S. Application Data
(63) Continuation of application No. 13/127,656.

(30) Foreign Application Priority Data
Nov. 4, 2008 (IN) 2351/MUM/2008
Nov. 14, 2008 (IN) 2402/MUM/2008
Oct. 29, 2009 (GB) PCT/GB2009/002575

(51) Int. Cl. A61K 47/48; A61M 15/00; B65B 1/02; A61M 11/04

(52) U.S. Cl. CPC A61K 47/48792; A61K 47/48176; A61M 11/04; A61M 15/00; B65B 1/02
USPC 128/200.23; 424/45; 424/400; 424/78.24; 525/326.7; 53/467

(57) ABSTRACT

A pharmaceutical aerosol composition comprising at least one hydrofluoroalkane propellant; at least one active agent complexed with an adjuvant; and, optionally, at least one pharmaceutically acceptable excipient.

### CROSS-REFERENCE TO RELATED APPLICATIONS

This application is a continuation of U.S. patent application Ser. No. 13/127,656 filed May 4, 2011 which is a filing under 35 U.S.C. 371 of International Application No. PCT/GB2009/002617 filed Nov. 4, 2009, entitled “Pharmaceutical Aerosol Composition,” claiming priority of International Application No. PCT/GB2009/002575 filed Oct. 29, 2009, and Indian Patent Application Nos. 2351/MUM/2008 filed Nov. 4, 2008, and 2402/MUM/2008 filed Nov. 14, 2008, which applications are incorporated by reference herein in their entirety.

### FIELD OF INVENTION

The present invention relates to a pharmaceutical aerosol composition. In particular, the present invention relates to a stable pharmaceutical aerosol composition, its process of manufacturing and its use for the treatment of respiratory disorders.

### BACKGROUND OF THE INVENTION

Metered dose inhalers (MDIs) are, at present, the most efficient and best-accepted means for accurately delivering drugs in small doses to the human respiratory tract. Therapeutic agents commonly delivered by the inhalation route include bronchodilators (β-2 agonists and anticholinergics), corticosteroids, and anti-allergics. Inhalation may also be a viable route for anti-infective, vaccinating, systemically acting and diagnostic agents, as well as anti-leukotrienes, and anti-proteases.

MDIs comprise a pressure resistant container typically filled with a product such as a drug dissolved in a liquified propellant or micronized particles suspended in a liquified propellant where the container is fitted with a metering valve. Actuation of the metering valve allows a small portion of the spray product to be released whereby the pressure of the liquified propellant carries the dissolved or micronized drug particles out of the container to the patient. The valve actuator is used to direct the aerosol spray into the patient's oropharynx.

Chlorofluorocarbons (CFCs) have been used extensively as propellants in drug formulations that are delivered to patients via a metered dose inhaler (MDI). However, recent scientific evidence suggests that CFCs damage the Earth's ozone layer. It is believed that ozone blocks harmful ultraviolet rays and that depletion of the ozone layer will result in the incidence of skin cancer. As a result, CFCs are now gradually being replaced by hydrofluorocarbons or HFAs (the names will be used interchangeably herein), (e.g., HFA 134a and HFA 227) as the preferred MDI propellants.

Propellant HFA 134a, or 1,1,1,2-tetrafluoroethane (the names will be used interchangeably herein) is non-flammable, has low toxicity, and has vapor pressure suitable for use in aerosols. However, HFA 134a is a very poor solvent which fails to dissolve or adequately disperse commonly used surfactants such as sorbitan trioleate, sorbitan monooleate, lecithins, and oleic acid in useful concentrations without the aid of a co-solvent.

Similarly, propellant HFA 227, or 1,1,1,2,3,3,3-heptafluoropropane (the names will be used interchangeably herein) is non-flammable, has low toxicity, and has a vapor pressure suitable for use in aerosols. However, the polarity and solubility of HFA 227 differ from those of commonly used CFC propellants, and many commonly used surfactants are not soluble or are poorly dispersible in HFA 227.

U.S. Pat. No. 5,182,097 relates to aerosol formulations consisting of 1,1,1,2-tetrafluoroethane, a drug and oleic acid as a surfactant to aid in dispersing the drug in the propellant.

U.S. Pat. No. 5,612,053 relates to respirable dry powder formulations comprising controlled release particles of a cohesive composite of a drug and a carrier, wherein the carrier is a natural polysaccharide gum added as a filler.

EP 0384371 discloses aerosols in which 1,1,1,2,3,3,3-heptafluoropropane or its mixture with propane, butane, isobutane, dimethyl ether, or 1,1, difluoroethane serves as the propellant. The application does not, however, disclose suspension aerosols or pharmaceutical aerosol formulation.

WO 93/11747 discloses a pharmaceutical suspension formulation suitable for aerosol administration, consisting essentially of a therapeutically effective amount of a drug and a propellant selected from the group consisting of HFA 134a, HFA 227, and a mixture thereof. The application specifically discloses formulations of Formoterol Fumarate in HFA 134a, HFA 227 and 1:1 mixtures of HFA 134a and HFA 227. The formulations do not contain surfactants or ethanol. It is stated that mixtures of HFA 134a and HFA 227 may be adjusted for density matching with the drug.

WO 93/11745 discloses pharmaceutical aerosol formulations, substantially free of surfactant containing fluorocarbon or hydrogen-containing chlorofluorocarbon propellants and up to 5% of a polar co-solvent. Preferred propellants are HFA 134a and HFA 227 which are preferably used alone. The preferred polar co-solvent is ethanol, and it is stated that in general only small quantities, e.g., 0.05 to 3.0% w/w of polar co-solvent are required to improve the dispersion and the use of quantities in excess of 5% w/w may disadvantageously tend to dissolve the medicament.

A number of medicinal aerosol formulations using HFA propellant systems are disclosed in, for example, EP 0372777, WO 91/11173, and WO 91/14422. These applications are all concerned with the preparation of pressurised aerosols for the administration of medicaments. All applications propose the addition of one or more of adjuvants such as alcohols, alkanes, dimethyl ether, surfactants (including fluorinated and non-fluorinated surfactants, carboxylic acids, polyethoxylates, etc.) and even conventional chlorofluorocarbon propellants in small amounts intended to minimise potential ozone damage, but have not been able to establish any unexpected advantage.

U.S. Pat. No. 6,303,103 discloses a pharmaceutical aerosol formulation which comprises particulate salbutamol and an anticholinergic medicament or physiologically acceptable salts and solvates thereof, a fluorocarbon or hydrogen-containing chlorofluorocarbon propellant and substantially free of or containing less than 0.0001% of surfactant by weight of the medicament. However, to support the claimed invention, there is no explicit demonstration by way of examples or any stability study data.

Attempts have also been made to coat drug with only surfactants or instead, but the results have not been very satisfactory.

WO 91/04011 describes a self-propelling powder aerosol composition containing finely-divided, pre-micronized solid drug coated with a single non-perfluorinated surface-active dispersing agent suspended in an aerosol propellant in which the dispersing agent is substantially insoluble.

PCT/GB2008/002029 describes a formulation comprising tiotropium bromide coated with polyvinylpyrrolidone. This coating approach suffers from the disadvantage that it requires multiple processing steps and may cause difficulties in scale-up due to overspray and lack of uniformity.

The disclosure of all these patents and patent applications are herein incorporated by reference.

Most of the above prior art suggests the use of surfactants. However, the use of surfactants may not be feasible for all drugs because considerable difficulties have been encountered in finding suitable suspending agents or surfactants which are soluble in hydrofluoroalkanes and capable of stabilizing medicament suspensions. Also, with additional incorporation of excipients along with the surfactants may destabilize the formulation.

Since the HFAs are more polar and more hygroscopic than CFCs, water ingress is more significantly found in HFA MDIs, and which also worsened by use of polar, water miscible cosolvents, such as alcohol. The above phenomenon could also be influenced by nature of the drug substance or other excipients present in the formulation. Ambient moisture diffuses into MDIs through the valve gaskets and adversely affects the physical stability of MDI formulation by promoting drug particle growth and aggregation. The aggregation of drug particles may clog the valve orifice of the aerosol container rendering the dispensing device inoperative, or if a metering valve is employed, it may be render inaccurate dosing which, in the case of highly potent drugs, may lead to undesirable results. In addition to increasing the particle size and clogging orifices, agglomeration may make the suspension physically unstable, an obviously undesirable result particularly in the case of aerosolized formulations.

Therefore, there still remains a need to provide a way to solve the problem of fine drug particle aggregation and provide a stable aerosol composition utilizing hydrofluorocarbon propellants.

### OBJECTS OF THE INVENTION

The object of the present invention is to provide a stable pharmaceutical aerosol composition.

Another object of the present invention is to provide a pharmaceutical aerosol composition wherein the fine drug particles do not aggregate during the storage.

Yet another object of the present invention is to provide a simple process to manufacture the pharmaceutical aerosol composition according to the present invention.

Still another object of the present invention is to provide a pharmaceutical aerosol composition with dose uniformity throughout the life of the can.

A further object of the present invention is to provide a pharmaceutical aerosol composition for use in the treatment of mild, moderate or severe acute or chronic symptoms or for prophylactic treatment of respiratory disorders.

### SUMMARY OF THE INVENTION

According to a first aspect of the present invention, there is provided a stable pharmaceutical aerosol composition comprising one or more HFA propellants and at least one active complexed with an adjuvant; and, optionally, one or more pharmaceutically acceptable excipient.

According to a second aspect of the present invention, there is provided a stable pharmaceutical aerosol composition comprising at least one active complexed with an adjuvant; one or more HFA propellants; and at least one other pharmaceutically acceptable excipient, such as at least one bulking agent and/or co-solvent.

According to a third aspect of the present invention, there is provided a process of manufacturing the said stable pharmaceutical aerosol composition.

According to a fourth aspect of the present invention, there is provided a pharmaceutical aerosol composition for use in the treatment of mild, moderate or severe acute or chronic symptoms, or for prophylactic treatment, of respiratory disorders such as asthma.

### DETAILED DESCRIPTION OF THE INVENTION

As discussed earlier, there is a need to provide a novel way to solve the problem of aggregation of fine drug particles of aerosol composition and which remains stable throughout the storage.

Surprisingly, the inventors have found that when the drug-adjuvant complex is used in the manufacture of aerosol composition, the aggregation of fine drug particles is significantly reduced, which renders the composition stable throughout the storage period.

The inventors further observed that the dispersion of surfactant in the pharmaceutical aerosol composition with other pharmaceutically acceptable excipients rendered the composition unstable during the storage. In particular, it was observed that the fine particle mass does not remain same or decreases in timely manner during the storage. But, it was surprisingly found that when the drug was complexed with an adjuvant such as PVP K 25, PVP K 17 or PVP K30 etc., along with the addition of propellant(s) or optionally with one or more bulking agent and/or co-solvent(s), aggregation of fine drug particles was reduced significantly and hence keeping the composition stable during the storage period. It was also found that the composition continued to exhibit uniform delivered dose characteristics throughout the life of the MDI.

The present invention provides a pharmaceutical aerosol composition. More specifically, the pharmaceutical aerosol composition comprises at least one pharmaceutical active agent complexed with an adjuvant, at least one hydrofluoroalkane (HFA) propellant and optionally other suitable excipients such as one or more bulking agent(s) and/or co-solvent(s).

In a particularly advantageous embodiment, the adjuvant is a polymer, preferably a polyvinylpyrrolidone (PVP and other commercially available grades such as PVP K12, PVP K15. PVP K17, PVP K25, PVP K30, PVP K60, PVP K90), preferably having a molecular weight ranging from PVP 2500 to PVP 1,200,000, preferably PVP 2500 to PVP 1,000,000. It has been found that when such polymers are complexed with drugs, they yield good quality pharmaceutical aerosol suspensions.

The term “adjuvant” is used throughout the description in broad sense to include not only polyvinylpyrrolidone (PVP) and its other commercially available grades such as PVP K12, PVP K15, PVP K17, PVP K25, PVP K30, PVP K60, PVP K90, but also suitable water soluble and water insoluble polymers or their mixtures, cyclodextrins, polyethylene glycols (e.g., PEG 4000 and PEG 6000) and surfactants such as sorbitan trioleate, sorbitan monooleate, tweens, e.g., tween 20, 40, 60, 80, 120, lipids, lecithin, oleic acid, citric acid, and polyoxyethylene-lauryl ether (e.g. Brij 30).

The water soluble polymers that can be used as an adjuvant, according to the present invention, comprise homopolymers and co-polymers of N-vinyl lactams, especially homopolymers and co-polymers of N-vinyl pyrrolidone, e.g., polyvinylpyrrolidone (PVP), co-polymers of PVP and vinyl acetate, co-polymers of N-vinyl pyrrolidone and vinyl acetate or vinyl propionate, dextrins such as grades of maltodextrin, cellulose esters and cellulose ethers, high molecular polyalkylene oxides such as polyethylene oxide and polypropylene oxide and co-polymers of ethylene oxide and propylene oxide and the like.

The water insoluble polymers that can be used as an adjuvant, according to the present invention, comprise acrylic copolymers, e.g., Eudragit E100 or Eudragit EPO; Eudragit L30D-55, Eudragit FS30D, Eudragit RL30D, Eudragit RS30D, Eudragit NE30D, Acryl-Eze; polyvinylacetate, for example, Kollicoat SR 30D; cellulose derivatives such as ethylcellulose, cellulose acetate and the like.

According to the present invention, the amount of adjuvant in the drug-adjuvant complex ranges from 0.5% to 500% by weight of the drug, preferably from 0.5% to 100% by weight of the drug. Suitable amounts of the adjuvant are 0.5%, 1%, 2%, 10%, 50% and 100% by weight of the drug.

The drug-adjuvant is in particulate (micronised form). Preferably, the particle size is such as to permit substantially all of the particles to be potentially available for inhalation into the lungs upon administration of the aerosol composition. Thus, for example, it is preferred that at least 90%, more preferably at least 95% by weight of the particles have a diameter of less than 15 micrometers, preferably from 0.5 to 15 micrometers, more preferably from 0.5 to 10 micrometers, and most preferably from 0.5 to 5 micrometers. Most preferably at least 95% by weight of the particles have a diameter from 0.5 to 5 micrometers.

According to the invention, drugs which may be complexed in aerosol compositions include any drug useful in inhalation therapy known to a person skilled in the art which comprises one or more of the following, for example, analgesics, e.g., codeine, dihydromorphine, ergotamine, fentanyl or morphine; anginal preparations, e.g., diltiazem; antiallergics, e.g., cromoglycate, ketotifen or nedocromil; anti-infectives, e.g., cephalosporins, penicillins, streptomycin, sulphonamides, tetracyclines and pentamidine; antihistamines, e.g., methapyrilene; anti-inflammatories, e.g., beclomethasone, flunisolide, budesonide, tipredane, triamcinolone acetonide, or fluticasone; corticosteroids e.g., budesonide, ciclesonide, fluticasone, betamethasone, beclomethasone, tixocortol, formocortal, rimexolone, prednisolone, methylprednisolone, deflazacort, prednisone, cortisone, dexamethasone, hydroocortisone; antitussives, e.g., noscapine; bronchodilators, e.g., ephedrine, adrenaline, fenoterol, formoterol, isoprenaline, metaproterenol, phenylephrine, phenylpropanolamine, pirbuterol, reproterol, rimiterol, salbutamol, salmeterol, terbutaline, isoetharine, tulobuterol, orciprenaline, or (−)4-amino-3,5-dichloro-.alpha.-[[[6-[2-(2-pyridinyl)ethoxy]hexyl]amino]methyl]benzenemethanol; diuretics, e.g., amiloride; anticholinergics, e.g., tiotropium, ipratropium, aclidinium, atropine or oxitropium; hormones, e.g., cortisone, hydrocortisone or prednisolone; xanthines, e.g., aminophylline, choline theophyllinate, lysine theophyllinate or theophylline; and therapeutic proteins and peptides, e.g., insulin or glucagon or pharmaceutically acceptable salts thereof.

It will be well appreciated to a person skilled in the art that the drugs may be used in the form of their pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs, pharmaceutically acceptable prodrugs thereof to optimize the activity and/or stability of the composition.

Particularly, preferred drugs for complexation in aerosol composition, according to the invention, include antiallergics, anticholinergics, bronchodilators and corticosteroids, for example, salbutamol, beclomethasone, ipratropium, formoterol, tiotropium, aclidinium, salmeterol, fluticasone, budesonide, fenoterol, ciclesonide, mometasone, or their pharmaceutically acceptable salts are especially preferred.

The terms “bronchodilators” or “corticosteroids” and the like are used in broad sense to include not only the beta-agonists or anticholinergic agents or corticosteroids per se, but also their pharmaceutically acceptable salts, pharmaceutically acceptable solvates, pharmaceutically acceptable hydrates, pharmaceutically acceptable enantiomers, pharmaceutically acceptable derivatives, pharmaceutically acceptable polymorphs and pharmaceutically acceptable prodrugs.

Where appropriate, the following salts of the drugs mentioned above may be used; acetate, benzenesulphonate, benzoate, bicarbonate, bitartrate, bromide, calcium edetate, camsylate, carbonate, chloride, citrate, dihydrochloride, edetate, edisylate, estolate, esylate, fumarate, fluceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylbromide, methylnitrate, methylsulphate, mucate, napsylate, nitrate, pamoate (embonate), pantothenate, phosphate diphosphate, polygalacturonate, salicylate, stearate, subacetate, succinate, sulphate, tannate, tartrate, and triethiodide.

Betamimetic agents useful in the composition of the present invention include, but are not limited to, salbutamol, formoterol, levalbuterol, carmoterol, pirbuterol and salmeterol. Suitable pharmaceutically acceptable salts of the betamimetics include, but are not limited to, the hydrochloride, sulfate, maleate, tartrate, and citrate salts. Preferably, the betamimetic agent is selected from salbutamol, salbutamol sulfate, levosalbutamol, levosalbutamol sulphate or levosalbutamol tartarate.

Anticholinergic agents useful in the composition of the present invention include, but are not limited to, oxitropium, ipratropium, aclidinium and tiotropium. Suitable pharmaceutically acceptable salts of the anticholinergic agents include, but are not limited to, the halide salts such as bromide, chloride or iodide. Preferably, the anticholinergic agent is tiotropium or tiotropium bromide or tiotropium bromide monohydrate.

A preferred pharmaceutical aerosol composition in accordance with the present invention comprises: (a) an effective amount of at least one drug complexed with an adjuvant; (b) at least one hydrofluorocarbon propellant; and (c) optionally, at least one pharmaceutically acceptable excipient including one or more bulking agent(s) and/or co-solvent(s).

In a particularly preferred embodiment of the present invention, there is provided a stable pharmaceutical aerosol composition comprising: (a) an effective amount of at least one pharmaceutical active selected from tiotropium, aclidinium, ipratropium, levosalbutamol or a pharmaceutically acceptable salt thereof complexed with PVP K17 or PVP K30; (b) at least one hydrofluorocarbon propellant; and (c) optionally at least one bulking agent(s) such as lactose and/or co-solvent(s) such as polyethylene glycol 400 (PEG 400) with other pharmaceutically acceptable excipients.

It will be appreciated by those skilled in the art that the aerosol composition according to the present invention may, if desired, comprise combination of two or more drugs. Accordingly, the present invention further provides aerosol composition comprising two or more drugs, wherein at least one of the drug is complexed with the adjuvant along with one or more optional pharmaceutically acceptable excipients.

Preferably, the pharmaceutical aerosol composition comprises bronchodilators such as tiotropium (e.g., as the bromide salt), salbutamol (e.g., as the free base or as the sulphate salt), levosalbutamol (e.g., as the sulphate salt), salmeterol (e.g., as the xinafoate salt), aclidinium, isoprenaline or their pharmaceutically acceptable salts or bronchodilator(s) in combination with one or more corticosteroid(s) such as a beclomethasone ester (e.g., the diproprionate), budesonide or a fluticasone ester (e.g., the propionate) or their other pharmaceutically acceptable salts, wherein at least one of the drug is complexed with the adjuvant.

Alternatively, the aerosol compositions may comprise a bronchodilator in combination with an antiallergic such as cromoglycate (e.g., the sodium salt). Examples of such combinations include isoprenaline and sodium cromoglycate; salmeterol and fluticasone propionate; or salbutamol and beclomethasone dipropionate and the like.

According to another aspect of the invention, there is provided a pharmaceutical aerosol dispenser comprising a canister having a chamber containing a pharmaceutical aerosol composition according to the present invention; an outlet for delivering the pharmaceutical aerosol composition to a patient in need thereof; and a valve for controlling flow of the pharmaceutical aerosol composition from the chamber to the outlet. Preferably, the canister is made up of metal, glass or a plastics material such as polysulphone plastics, for example, polysulphone (PSU) and polyethersulphone (PES). Preferably, the pharmaceutical aerosol device is a MDI.

The present invention further provides a process of manufacturing a pharmaceutical aerosol dispenser for delivering the aerosol pharmaceutical formulation to a patient in need thereof, comprising:

(a) weighing the complexed drug particles in a suitable metal canister,

(b) optionally mixing the complexed drug particles with one or more suitable excipients selected from cosolvents, bulking agents, antioxidants, lubricants and optionally with one or more surfactants or with other similarly complexed or non-complexed drugs, and

(c) crimping the canister with a suitable valve and charging with HFA Propellant.

In another aspect, the present invention provides a process of manufacturing a complex of an active agent and an adjuvant comprising:

(a) mixing the active agent in an organic solvent (e.g., acetone),

(b) heating the mixture from step (a) to a suitable temperature and adding water to form a clear solution;

(c) adding the adjuvant to the above solution from step (b);

(d) concentrating the clear solution under reduced pressure, preferably under vacuum, to form a residue;

(e) washing the residue with the same solvent used in step (a); and

(f) drying (for example at suitable temperature, or preferably 50° C.) the washed residue from step (e) to form a drug-adjuvant complex.

The solvent used in the above process may be selected from acetonitrile, methanol, water, dimethyl formamide, acetone, tetrahydrofuran, dimethyl sulfoxide. Most preferable solvent is acetone.

Alternatively, the complex of an active agent and an adjuvant can be isolated by lyophilization or by flash-evaporating the solvent using suitable techniques known in the art such as spray-drying. Flash-evaporating technique with respect to the present invention means removal of the solvent by applying heat and vacuum.

The active agent used in the process of preparing the complex with an adjuvant according to the present invention may be in amorphous, crystalline, monohydrate or anhydrous form or a derivative or a polymorph or a prodrug thereof.

The pharmaceutical aerosol composition according to the present invention may optionally contain one or more excipients or carriers conventionally used in the art of pharmaceutical aerosol formulation. Such optional excipients include, but are not limited to, taste masking agents, buffers, antioxidants, water, and chemical stabilizers.

Examples of suitable bulking agents include, but are not limited to, one or more of saccharides such as monosaccharides, disaccharides, polysaccharides, and sugar alcohols such as arabinose, glucose, fructose, ribose, mannose, sucrose, trehalose, lactose, maltose, starches, dextran, or mannitol. The bulking agent may be present in a concentration of 0.005-500%, more preferably in a range of 0.005-300% by weight of the complex. The preferred bulking agent is Lactose.

Examples of cosolvents include, but are not limited to, one or more of polyethylene glycol (“PEG”), propylene glycol, isopropyl myristate or glycerol. Suitably, the cosolvent is PEG, such as PEG 200 or PEG 400. The cosolvent can be present in a range of about 0.05% to about 15% by weight of the composition. Suitably, the cosolvent is present in a range of about 0.05% to about 1% or about 0.05% to about 0.3% by weight of the composition.

The present invention may optionally comprise antioxidants like citric acid, benzalkonium chloride.

As discussed earlier, the aerosol compositions traditionally contained CFC propellants. Due to environmental concerns, HFA propellants are now preferred over CFC propellants. As will be understood by those skilled in the art, suitable HFA propellants for use in the present invention include, but are not limited to, 1,1,1,2-tetrafluoroethane (HFA-134a) and 1,1,1,2,3,3,3-heptafluoropropane (HFA-227). Optionally mixtures of two or more such halogen-substituted hydrocarbons may also be used.

The invention is particularly useful in that it allows pharmaceutically acceptably stable dispersions to be attained using HFA propellant as the aerosol propellant. The aerosol compositions of the invention may be prepared with HFA propellant alone or a mixture of HFA propellant and another miscible adjuvant having a polarity equal to or lower than the polarity of the HFA propellant.

In a further aspect, the present invention provides a pharmaceutical aerosol composition according to the present invention for use in the treatment of mild, moderate or severe acute or chronic symptoms, or for prophylactic treatment, of a respiratory disorder, such as asthma.

In a preferred embodiment, the present invention provides use of the pharmaceutical aerosol composition according to the present invention in the manufacture of a medicament for the treatment of mild, moderate or severe acute or chronic symptoms, or for prophylactic treatment, of a respiratory disorder, such as asthma.

In another preferred embodiment, there is provided a method of treatment of mild, moderate or severe acute or chronic symptoms, or for prophylactic treatment, of a respiratory disorder, such as asthma, comprising administering a therapeutically effective amount of a pharmaceutical aerosol composition according to the present invention to a patient in need thereof.

### EXAMPLES

The following examples are for the purpose of illustration of the invention only and are not intended in any way to limit the scope of the present invention. Process of preparing the tiotropium bromide-PVP complex

1) 5 g of Tiotropium bromide was introduced in a reaction vessel. Acetone was added. The reaction mixture was heated to a temperature of 50-55° C. Further water (15 ml) was added to the reaction mixture to get a clear solution. To this solution 2.5 g of PVP-K-17 was added. The solution was concentrated under vacuum to get a residue. The residue was washed with acetone (15 ml) and dried under vacuum at 50° C. to get the title compound (6 g).

2) 5 g of Tiotropium bromide was introduced in a reaction vessel. Water (15 ml) was added to the reaction mixture to get a clear solution. To this solution 2.5 g of PVP-K-17 was added. The solution was concentrated under vacuum to get a residue. The residue was washed with acetone (15 ml) and dried under vacuum at 50° C. to get the title complex (6 g).

3) Tiotropium Bromide-PVP Complex Formed by Lyophilization

5 g of Tiotropium bromide and 2.5 g of polyvinylpyrrolidone (PVP) was stirred in 50 ml of water at 25-30° C. until dissolved. The solution was quick freezed in a dry ice bath. The solution was lyophilized at a condenser temperature of approximately 0° C. in presence of high vacuum. The resultant title complex was obtained in the form of a solid (5.8 g).

4) Tiotropium Bromide-PVP Complex Formed by Evaporation

2.5 g of Tiotropium bromide and 1.25 g of polyvinylpyrrolidone (PVP) was stirred in 10 ml of ethanol at 25-30° C. until dissolved. Pour the solution into a polyethylene tray and evaporate the ethanol in a vacuum oven in presence of a nitrogen stream. The resultant dry solid title product was isolated (2.5 g).

5) Tiotropium bromide-PVP Complex Formed by Spray Drying 2.5 g of Tiotropium bromide was dissolved in 25 ml of methanol. 1.25 g of PVP was dissolved in methanol (15 ml). Both the solutions were mixed and spray dried. Parameters used for the manufacture of the drug-adjuvant complex are as follows:

Instrument—Labultima LU-222 ADVANCE SPRAY DRYER

Temperature—50-55° C.

Vacuum—40 mmWC

N₂ pressure—1-2 kg

Feeding rate—3ml/min.

The solid product was collected in a collector (2.0 g).

### Examples of Pharmaceutical Aerosol Composition:

Composition 1: Pharmaceutical aerosol composition containing Tiotropium-PVP complex (0.5%), co-solvent and HFA propellant.

| Ingredients | Qty/can |
|---|---|
| Tiotropium monohydrate or | 1.8 mg |
| anhydrous-PVP complex (0.5%) | |
| PEG 400 | 33.6 mg |
| HFA-227 | Q.S. |

### Process:

(a) The active ingredient was added to the canister.
(b) The canister was crimped with the metered valve and was charged with the solution of propellant and PEG.

Composition 2: Pharmaceutical aerosol composition containing Tiotropium-PVP complex (100%), co-solvent and HFA propellant.

| Ingredients | Qty/can |
|---|---|
| Tiotropium monohydrate or | 3.6 mg |
| anhydrous -PVP complex (100%) | |
| PEG 400 | 33.6 mg |
| HFA-227 | Q.S. |

### Process:

(a) The active ingredient was added to the canister.
(b) The canister was crimped with the metered valve and was charged with the solution of propellant and PEG.

Composition 3: Pharmaceutical aerosol composition containing Tiotropium-PVP complex (0.5%) and HFA propellant.

| Ingredients | Qty/can |
|---|---|
| Tiotropium monohydrate or | 1.8 mg |
| anhydrous-PVP complex (0.5%) | |
| HFA-227 | Q.S. |

### Process:

(a) The active ingredient was added to the canister.
(b) The canister was crimped with the metered valve.
(c) The canister was charged with HFA227.

Composition 4: Pharmaceutical aerosol composition containing Ipratropium bromide-PVP complex (0.5%) and HFA propellant.

| Ingredients | Qty/can |
|---|---|
| Ipratropium Bromide monohydrate | 5.04 mg |
| or anhydrous-PVP complex (0.5%) | |
| HFA-227 | Q.S. |

### Process:

(a) The active ingredient was added to the canister.
(b) The canister was crimped with the metered valve.
(c) The canister was charged with HFA227.

Composition 5: Pharmaceutical aerosol composition containing Tiotropium-PVP complex (100%) and HFA propellant.

| Ingredients | Qty/can |
|---|---|
| Tiotropium monohydrate or | 3.6 mg |
| anhydrous-PVP complex (100%) | |
| HFA-227 | Q.S. |

### Process:

(a) The active ingredient was added to the canister.
(b) The canister was crimped with the metered valve.
(c) The canister was charged with HFA227.

Composition 6: Pharmaceutical aerosol composition containing Tiotropium-PVP complex (0.5%), bulking agent, co-solvent and HFA propellant.

| Ingredients | Qty/can |
|---|---|
| Tiotropium monohydrate or | 1.8 mg |
| anhydrous-PVP complex (0.5%) | |
| Lactose | 1.8 mg |
| HFA-227 | Q.S. |

### Process:

(a) The active ingredient to the canister.
(b) Lactose was added to (a).
(c) The canister was crimped with the metered valve and was charged with propellant.

Composition 7: Pharmaceutical aerosol composition containing Levosalbutamol sulphate-PVP complex (0.5%), bulking agent and HFA propellant.

| Ingredients | Qty/can |
|---|---|
| Levosalbutamol Sulphate-PVP | 14.4 mg |
| complex (0.5%) | |
| Lactose | 1.8 mg |
| HFA-227 | Q.S. |

### Process:

(a) The active ingredient to the canister.
(b) Lactose was added to (a).
(c) The canister was crimped with the metered valve and was charged with propellant.

Composition 8: Pharmaceutical aerosol composition containing Levosalbutamol tartarate-PVP complex (0.5%), bulking agent and HFA propellant.

| Ingredients | Qty/can |
|---|---|
| Levosalbutamol Tartrate-PVP | 15.6 mg |
| complex (0.5%) | |
| Lactose | 1.8 mg |
| HFA-227 | Q.S. |

### Process:

(a) The active ingredient to the canister.
(b) Lactose was added to (a).
(c) The canister was crimped with the metered valve and was charged with propellant.

Composition 9: Pharmaceutical aerosol composition containing Tiotropium-PVP complex (0.5%), bulking agent, co-solvent and HFA propellant.

| Ingredients | Qty/can |
|---|---|
| Tiotropium monohydrate or | 1.8 mg |
| anhydrous-PVP complex (0.5%) | |
| Lactose | 1.8 mg |
| PEG 400 | 33.6 mg |
| HFA-227 | Q.S. |

### Process:

(a) The active ingredient was added to the canister.
(b) Lactose was added to (a).
(c) The canister was crimped with the metered valve and was charged with the solution of propellant and PEG.

Composition 10: Pharmaceutical aerosol composition containing Tiotropium-PVP complex (0.5%), bulking agent, co-solvent and HFA propellant.

| Ingredients | Qty/can |
|---|---|
| Tiotropium monohydrate or | 1.8 mg |
| anhydrous-PVP complex (0.5%) | |
| Lactose | 1.8 mg |
| PEG 1000 | 11.2 mg |
| HFA-227 | Q.S. |

### Process:

(a) The active ingredient was added to the canister.
(b) Lactose was added to (a).
(c) The canister was crimped with the metered valve and was charged with the solution of propellant and PEG.

Composition 11: Pharmaceutical aerosol composition containing Tiotropium-PVP complex (0.5%), bulking agent, co-solvent and HFA propellant.

| Ingredients | Qty/can |
|---|---|
| Tiotropium monohydrate or anhydrous- | 1.8 mg |
| PVP complex (0.5%) | |
| Lactose | 5.4 mg |
| PEG 1000 | 11.2 mg |
| HFA-227 | Q.S. |

### Process:

(a) The active ingredient was added to the canister.
(b) Lactose was added to (a).
(c) The canister was crimped with the metered valve and was charged with the solution of propellant and PEG.

Composition 12:

| Ingredients | Qty/can |
|---|---|
| Tiotropium monohydrate or | 3.6 mg |
| anhydrous-PVP complex (0.5%) | |
| Ciclesonide | 32 mg |
| Formoterol | 0.96 mg |
| HFA-227 | Q.S. |

### Process:

(a) The active ingredients were added to the canister.
(b) The canister was crimped with the metered valve.
(c) The canister was charged with HFA227.

### Composition 13:

| Ingredients | Qty/can |
|---|---|
| Levosalbutamol-PVP complex | 15.6 mg |
| (0.5%) | |
| Lactose | 12.5 mg |
| HFA-227 | Q.S. |

### Process:

(a) The active ingredient is added to the canister.
(b) The canister was crimped with the metered valve.
(c) The canister was charged with HFA227.

Composition 14:

| Ingredients | Qty/can |
|---|---|
| Levosalbutamol-PVP complex | 15.6 mg |
| (0.5%) | |
| Ipratropium Bromide | 5.04 mg |
| Lactose | 12.5 mg |
| HFA-227 | Q.S. |

### Process:

(a) The active ingredients were added to the canister.
(b) The canister was crimped with the metered valve.
(c) The canister was charged with HFA227.

Composition 15:

| Ingredients | Qty/can |
|---|---|
| Salbutamol sulphate-PVP complex | 28.8 mg |
| (0.5%) | |
| Ipratropium-PVP complex (0.5%) | 5.04 mg |
| Lactose | 12.5 mg |
| HFA-227 | Q.S. |

### Process:

(a) The active ingredients were added to the canister.
(b) The canister was crimped with the metered valve.
(c) The canister was charged with HFA227.

Composition 16:

| Ingredients | Qty/can |
|---|---|
| Salbutamol sulphate-PVP complex | 28.8 mg |
| (0.5%) | |
| Ipratropium-PVP complex (0.5%) | 5.04 mg |
| PEG 400 | 0.1 mg |
| Lactose | 12.5 mg |
| HFA-227 | Q.S. |

### Process:

(a) The active ingredients were added to the canister.
(b) The canister was crimped with the metered valve and the canister was charged with the solution of propellant and PEG.

### Stability Study Data of Pharmaceutical Aerosol Composition Comprising Drug-Adjuvant Complex and HFA:

An accelerated stability study was carried out comparing following three different set of compositions (1) drug and HFA propellant; (2) drug-adjuvant complex and HFA propellant; & (3) drug, co-solvent, surfactant, bulking agent and HFA propellant.

| Composition | | | | Fine Particle |
|---|---|---|---|---|
| No. | Ingredients | Qty/Can (mg) | Results | mass (mcg) |
| 1 | Tiotropium Bromide | 1.8 | Initial | 3.6 |
| | monohydrate | | | |
| | HFA 227 | Q.S. | 3 months, 40° C. | 2.1 |
| | | | 6 months, 40° C. | 1.4 |
| 2 | Tiotropium Bromide | 2.7 | Initial | 4.2 |
| | monohydrate-PVP-K-17 | | | |
| | complex (50%) | | | |
| | HFA 227 | Q.S. | 3 months, 40° C. | 4.3 |
| | | | 6 months, 40° C. | 4.1 |
| 3 | Tiotropium Bromide | 1.8 | Initial | 4.0 |
| | monohydrate-PVP-K-25 | | | |
| | complex (0.5%) | | | |
| | HFA 227 | Q.S. | 3 months, 40° C. | 4.2 |
| | | | 6 months, 40° C. | 4.1 |
| 4 | Tiotropium Bromide | 1.8 | Initial | 3.9 |
| | monohydrate | | | |
| | PEG 400 | 0.3% | 3 months, 40° C. | 3.9 |
| | PVP-K-25 | 0.001% | 6 months, 40° C. | 4.5 |
| | HFA 227 | Q.S. | | |

It was found that the aerosol composition containing drug-adjuvant complex exhibited consistent fine particle size during the study when stored at accelerated stability conditions.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a propellant” includes a single propellant as well as two or more different propellants; reference to a “cosolvent” refers to a single cosolvent or to combinations of two or more cosolvents, and the like.

1. A pharmaceutical aerosol composition comprising at least one hydrofluoroalkane (HFA) propellant; at least one active agent complexed with an adjuvant; and, optionally, one or more pharmaceutically acceptable excipients.

2. The pharmaceutical aerosol composition according to claim 1, wherein the adjuvant is selected from polymer; cyclodextrins, polyethylene glycols, lipids, citric acid, and surfactants such as sorbitan trioleate, sorbitan monooleate, tweens, lecithin, oleic acid, polyoxyethylene-lauryl ether, or mixtures thereof.

3. The pharmaceutical aerosol composition according to claim 2, wherein the adjuvant is a polymer.

4. The pharmaceutical aerosol composition according to claim 3, wherein the polymer is a water soluble polymer, a water insoluble polymer; or a mixture thereof.

5. The pharmaceutical aerosol composition according to claim 4, wherein the water soluble polymer comprises polyvinylpyrrolidone, homopolymers and co-polymers of N-vinyl lactams such as homopolymers and co-polymers of N-vinyl pyrrolidone, co-polymers of polyvinyl pyrrolidone and vinyl acetate, co-polymers of N-vinyl pyrrolidone and vinyl acetate or vinyl propionate, dextrins such as grades of maltodextrin, cellulose esters and cellulose ethers, high molecular polyalkylene oxides such as polyethylene oxide and polypropylene oxide and co-polymers of ethylene oxide and propylene oxide.

6. The pharmaceutical aerosol composition according to claim 4, wherein the water insoluble polymer comprises acrylic copolymers; polyvinylacetate; cellulose derivatives such as ethylcellulose, cellulose acetate.

7. The pharmaceutical aerosol composition according to claim 1, wherein the adjuvant is a polyvinylpyrrolidone.

8. The pharmaceutical aerosol composition according to claim 7, wherein the polyvinylpyrrolidone is a polyvinylpyrrolidone having a molecular weight ranging from 2500 to 1,200,000.

9. The pharmaceutical aerosol composition according to claim 1, wherein the amount of adjuvant in the complex of the active agent and the adjuvant ranges from 0.5% to 500% by weight of the active agent.

10. The pharmaceutical aerosol composition according to claim 1, wherein the complex of the active agent and the adjuvant is particulate and has a size distribution such that at least 90% of the particles have a diameter of less than or equal 15 micrometers.

11. The pharmaceutical aerosol composition according to claim 1, wherein the active agent is an antiallergic, an anticholinergic or a bronchodilator.

12. The pharmaceutical aerosol composition according to claim 11, wherein the active agent is selected from tiotropium, salbutamol, beclomethasone, ipratropium, formoterol, aclidinium, salmeterol, fluticasone, budesonide, fenoterol, ciclesonide, mometasone, or a pharmaceutically acceptable salt thereof.

13. The pharmaceutical aerosol composition according to claim 12, wherein the active agent is salbutamol, salbutamol sulfate, levosalbutamol, levosalbutamol sulphate, levosalbutamol tartarate, ipratropium, ipratropium bromide, tiotropium, tiotropium bromide or tiotropium bromide monohydrate.

14. The pharmaceutical aerosol composition according to claim 1, wherein the pharmaceutically acceptable excipients include a bulking agent, a cosolvent or both.

15. The pharmaceutical aerosol composition according to claim 14, wherein the pharmaceutically acceptable excipients further include a taste masking agent, a buffer, an antioxidant, water, and a chemical stabilizer.

16. The pharmaceutical aerosol composition according to claim 14, wherein the bulking agent includes one or more saccharides and/or one or more sugar alcohols.

17. The pharmaceutical aerosol composition according to claim 14, wherein the cosolvent includes one or more of polyethylene glycol, propylene glycol, isopropyl myristate and glycerol.

18. The pharmaceutical aerosol composition according to claim 1, wherein the HFA propellant is 1,1,1,2-tetrafluoroethane (HFA-134a), 1,1,1,2,3,3,3-heptafluoropropane (HFA-227) or a mixture thereof.

19. The pharmaceutical aerosol composition according to claim 1 for use in the treatment of mild, moderate or severe acute or chronic symptoms, or for prophylactic treatment, of a respiratory disorder, such as asthma.

20. A method comprising utilizing the pharmaceutical aerosol composition according to claim 1 in the manufacture of a medicament for the treatment of mild, moderate or severe acute or chronic symptoms, or for prophylactic treatment, of a respiratory disorder, such as asthma.

21. A method of treatment of mild, moderate or severe acute or chronic symptoms, or for prophylactic treatment, of a respiratory disorder, such as asthma, comprising administering a therapeutically effective amount of a pharmaceutical aerosol composition according to claim 1 to a patient in need thereof.

22. A pharmaceutical aerosol dispenser comprising a canister having a chamber containing a pharmaceutical aerosol composition according to claim 1; an outlet for delivering the pharmaceutical aerosol composition to a patient in need thereof; and a valve for controlling flow of the pharmaceutical aerosol composition from the chamber to the outlet.

23. A process for manufacturing a pharmaceutical aerosol dispenser for delivering a pharmaceutical composition according to claim 1, comprising:
(a) placing a predetermined amount of the complex of the active agent and the adjuvant in a suitable canister,
(b) optionally mixing the complex with one or more pharmaceutically acceptable excipients; and
(c) crimping the canister with a suitable valve and charging with the HFA propellant.

24. A process for manufacturing a complex of an active agent and an adjuvant, comprising:
(a) mixing the active agent in an organic solvent;
(b) heating the mixture from step (a) to a suitable temperature and adding water to form a clear solution;
(c) adding the adjuvant to the solution from step (c);
(d) concentrating the solution from step (d) under reduced pressure to form a residue;
(e) washing the residue from step (d) with the same solvent used in step (a); and
(f) drying the washed residue from step (e) to form a drug-adjuvant complex.
